(19) United States
(12) Patent Application Publication
(10) Pub. No.: US 2006/0168667 A1
(43) Pub. Date: Jul. 27, 2006

(54) Minibrain homologous proteins involved in the regulation of energy homeostasis

(76) Inventors: Karsten Eulenberg, Bovenden (DE); Gunter Bronner, Bochum (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/523,643

(22) PCT Filed: Sep. 3, 2003
(86) PCT No.: PCT/EP03/09774

§ 371 Date: Feb. 4, 2005

(30) Foreign Application Priority Data
Sep. 3, 2002 (EP) 02 019-747.1

(51) Int. Cl. A01K 67/00; G01N 33/53; A61K 48/00; C12N 5/08

(52) U.S. Cl. 800/8; 424/93.21; 514/12; 435/366; 435/7.1

(57) ABSTRACT

The present invention discloses minibrain homologous proteins regulating the energy homeostasis and the metabolism of triglycerides, and polynucleotides, which identify and encode the proteins disclosed in this invention. The invention also relates to the use of these sequences in the diagnosis, study, prevention; and treatment of metabolic diseases and disorders.

This invention relates to the use of nucleic acid sequences encoding minibrain homologous proteins, to the use of polypeptides encoded thereby, and to the use of modulators/effectors of the proteins and polynucleotides in the diagnosis, study, prevention, and treatment of diseases and disorders related to body-weight regulation, for example, but not limited to, metabolic diseases or dysfunctions such as obesity, diabetes, and/or metabolic syndrome, as well as related disorders such as eating disorder, cachexia, hypertension, coronary heart disease, hypercholesterolemia, dyslipidemia, osteoarthritis, gallstones, or liver fibrosis.

There are several metabolic diseases of human and animal metabolism, e.g., obesity and severe weight loss, that relate to energy imbalance where caloric intake versus energy expenditure is imbalanced. Obesity is one of the most prevalent metabolic disorders in the world. It is still a poorly understood human disease that becomes more and more relevant for western society. Obesity is defined as a body weight more than 20% in excess of the ideal body weight, frequently resulting in a significant impairment of health. Obesity may be measured by body mass index, an indicator of adiposity or fatness. Further parameters for defining obesity are waist circumferences, skinfold thickness and bioimpedance. Obesity is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors, and can be caused by different reasons such as non-insulin dependent diabetes, increase in triglycerides, increase in carbohydrate bound energy and low energy expenditure. As such, it is a complex disorder that must be addressed on several fronts to achieve lasting positive clinical outcome. Since obesity is not to be considered as a single disorder but a heterogeneous group of conditions with (potential) multiple causes, it is also characterized by elevated fasting plasma insulin and an exaggerated insulin response to oral glucose intake (Koltermann O. G., (1980) J. Clin. Invest 65: 1272-1.284). A clear involvement of obesity in type 2 diabetes mellitus can be confirmed (Kopelman P. G., (2000) Nature 404: 635-643).

Hyperlipidemia and elevation of free fatty acids correlate clearly with the metabolic syndrome, which is defined as the linkage between several diseases, including obesity and insulin resistance. This often occurs in the same patients and are major risk factors for development of type 2 diabetes and cardiovascular disease. It was suggested that the control of lipid levels and glucose levels is required to treat type 2 diabetes, heart disease, and other occurances of metabolic syndrome (see, for example, Santomauro A. T. et al., (1999) Diabetes, 48: 1836-1841 and Lakka H. M., (2002) JAMA 288: 2709-2716).

The concept of ‘metabolic syndrome’ (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M. et al., (1988) Diabetes, 37: 1595-1607). Today metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type II diabetes and cardiovascular disease (Lakka H. M., 2002, supra).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms or/and molecules influencing obesity or body weight/body mass regulations are not known. In addition, several single-gene mutations resulting in obesity have been described in mice, implicating genetic factors in the etiology of obesity (Friedman J. M. and Leibel R. L., (1992), Cell 69: 217-220). In the obese (ob) mouse a single gene mutation (obese) results in profound obesity, which is accompanied by diabetes (Friedman J. M. et. al., (1991) Genomics 11: 1054-1062).

Therefore, the technical problem underlying the present invention was to provide for means and methods for modulating (pathological) metabolic conditions influencing body-weight regulation or/and energy homeostatic circuits. The solution to said technical problem is achieved by providing the embodiments characterized in the claims.

Accordingly, the present invention relates to novel functions of proteins and nucleic acids encoding these in body-weight regulation, energy homeostasis, metabolism, and obesity. The proteins disclosed herein and polynucleotides encoding these are thus suitable to investigate metabolic diseases and disorders. Further new compositions are provided that are useful in diagnosis, treatment, and prognosis of metabolic diseases and disorders as described.

In a first aspect, the present invention relates to a pharmaceutical composition comprising a minibrain homologous protein or/and a functional fragment thereof, a nucleic acid molecule encoding a minibrain homologous protein and/or a functional fragment thereof and/or a modulator/effector of said nucleic acid molecule and/or said protein together with pharmaceutically acceptable carriers, diluents and/or additives. In particular, the nucleic acid molecule is a vertebrate or insect minibrain nucleic acid, particulary encoding a human protein as described in Table 1, and/or a nucleic molecule which is complementary thereto or a functional fragment thereof or a variant thereof.

So far, it has not been described that a protein of the invention or a homologous protein is involved in the regulation of energy homeostasis and body-weight regulation and related disorders, and thus, no functions in metabolic diseases and dysfunctions and other diseases as listed above have been discussed. In this invention, we demonstrate that the correct gene dose of a protein of the invention is essential for maintenance of energy homeostasis. A genetic screen was used to identify that mutations of the gene encoding a protein of the invention causes changes in the metabolism, in particular related to obesity, which is reflected by a significant change in the triglyceride content, the major energy storage substance.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that minibrain (GadFly Accession Number CG7826) homologous proteins (herein referred to as “proteins of the invention” or “a protein of the invention”) are regulating the energy homeostasis and fat metabolism, especially the metabolism and storage of triglycerides, and polynucleotides, which identify and encode the proteins disclosed in this invention. The invention also relates to vectors, host cells, and recombinant methods for producing the polypeptides and polynucleotides of the invention. The invention also relates to the use of these compounds and modulators/effectors thereof, e.g. antibodies, biologically active nucleic acids, such as antisense molecules, RNAi molecules or ribozymes, aptamers, peptides or low-molecular weight organic compounds recognizing said polynulceotides or polyeptides, in the diagnosis, study, prevention, and treatment of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

The minibrain gene encodes a new family of protein kinases that is evolutionarily conserved from insects to humans. The minibrain gene of Drosophila melanogaster encodes a serine-threonine protein kinase with an essential role in post-embryonic neurogenesis. The human ortholog protein DYRK1A (dual specificity Yak 1-related kinase 1A) is a proline-directed and DYRK1B is an arginine-directed serine/threonine kinase. The involvement of

Dyrk1a in some of the neurological defects of Trisomy 21 or Down's syndrome patients was suggested because Dyrk1a has been mapped within the Down's Syndrome (DS) critical region of chromosome 21 and is overexpressed in DS embryonic brain. Down's syndrome is a major cause of mental redardation, hypotonia and delayed development (see also U.S. Pat. No. 6,251,664). Mice deficient in Dyrk1A function presented a general growth delay and died during midgestation. Mice heterozygous for the mutation (Dyrk1A(+/−)) showed decreased neonatal viability and a significant body size reduction from birth to adulthood. The data shown by Fotaki V. et al., (2002) Mol Cell Biol. 22: 6636-6647, suggest a conserved mode of action that determines normal growth and brain size in both mice and flies. Transgenic mice overexpressing Dyrk1a exhibit delayed cranio-caudal maturation with functional consequences in neuromotor development. Spatial learning and cognitive flexibility, indicative of hippocampal and prefrontal cortex dysfunction, are severe impaired (Altafaj X. et al., (2001) Hum Mol Genet. 10: 1915-1923).

Although no clear biological function has been described for the human orthologs of minibrain, it has recently been suggested Dyrk1A may function as a regulator controlling the assembly of endocytic apparatus. It was suggested that Dyrk1A might play a dual role in regulating the interaction of dynamin 1 with amphiphysin 1. Phosphorylation of Dyrk1A also reduced the interaction of dynamin with endophilin 1, whereas the same phosphorylation enhanced the binding of dynamin 1 the adaptor protein Grb2 (Chen-Hwang M. C. et al., (2002) J Biol Chem. 277: 17597-17604).

Dyrk1a has also been described to play a role in the phosphorylation of a variety of substrates, including the transcription factor Forkhead in rhabdomyosarcoma (FKHR), which has been implicated in the control of gene expression by insulin as well as the regulation of apoptosis by survival factors. (Woods Y. L. et al., (2001) Biochem J. 355: 597-607).

Dyrk1a is necessary for the glycogen synthase kinase 3 (GSK3) cophosphorylation (priming) of GSK3 target proteins (Woods Y. L. et al., (2001) Biochem J. 355: 609-615).

Dyrk1b is a substrate of mitogen-activated protein kinase. Activated extracellular signal-regulated kinases (erks) down regulate protein levels of Dykr1b, and the protein levels of Dyrk1b were increased 20-fold when erk activation was blocked (Lee K. et al., (2000) Cancer Res. 60: 3631-3637). The MAPK kinase MKK3 has been shown to activate Dyrk1b as protein kinase (Lim S. et al., (2002) Biol Chem. 277: 25040-25046).

A different member of the Saccharomyces cereviseae Dyrk family, called Yak1p, has been described to be involved in the phosphorylation of the glucose response transcription factor Pop2p. Pop2p controls cell growth (division) in response to the amount of glucose (glucose-sensing system) (Moriya H. et al., (2001) Genes Dev. 15(10): 1217-1228).

Minibrain homologous proteins and nucleic acid molecules coding therefore are obtainable from insect or vertebrate species, e.g. mammals or birds. Particularly preferred are homologous nucleic acids, particularly nucleic acids encoding a human protein as described in Table 1 or/and an isoform, fragment or variant of said protein.

The invention particularly relates to a nucleic acid molecule encoding a polypeptide contributing to regulating the energy homeostasis and the metabolism of triglycerides, wherein said nucleic acid molecule comprises
(a) the nucleotide sequence encoding Drosophila minibrain, or a human protein particularly as described in Table 1, and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of the minibrain protein, preferably of the human homologous proteins, particularly a human protein as described in Table 1,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of at least 15 bases, preferably at least 20 bases, more preferably at least 25 bases and most preferably at least 50 bases, in particular 15-25 bases, preferably 25-35 bases, more preferably 35-50 bases and most preferably at least 50 bases.

The invention is based on the finding that minibrain and/or homologous proteins and the polynucleotides encoding these, are involved in the regulation of triglyceride storage and therefore energy homeostasis. The invention describes the use of these compositions for the diagnosis, study, prevention, or treatment of metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, liver fibrosis, or gallstones.

Accordingly, the present invention relates to genes with novel functions in body-weight regulation, energy homeostasis, metabolism, and obesity, functional fragments of said genes, polypeptides encoded by said genes or functional fragments thereof, and modulators/effectors thereof, e.g. antibodies, biologically active nucleic acids, such as antisense molecules, RNAi molecules, or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

In one embodiment, a forward genetic screen is performed in fly displaying a mutant phenotype due to misexpression of a known gene (see, St Johnston D., (2002) Nat Rev Genet 3: 176-188; Rorth P., (1996) Proc Natl Acad Sci USA 93: 12418-12422). In this invention, we have used a genetic screen to identify mutations that cause changes in the body weight, which are reflected by a significant change of triglyceride levels.

Obese patients mainly show a significant increase in the content of triglycerides. Triglycerides are the most efficient storage for energy in cells. In order to isolate genes with a function in energy homeostasis, several thousand EP-lines were tested for their triglyceride content after a prolonged feeding period (see Examples for more detail). Lines with significantly changed triglyceride content were selected as positive candidates for further analysis. The change of triglyceride content due to the loss of a gene function suggests gene activities in energy homeostasis in a dose dependent manner that controls the amount of energy stored as triglycerides.

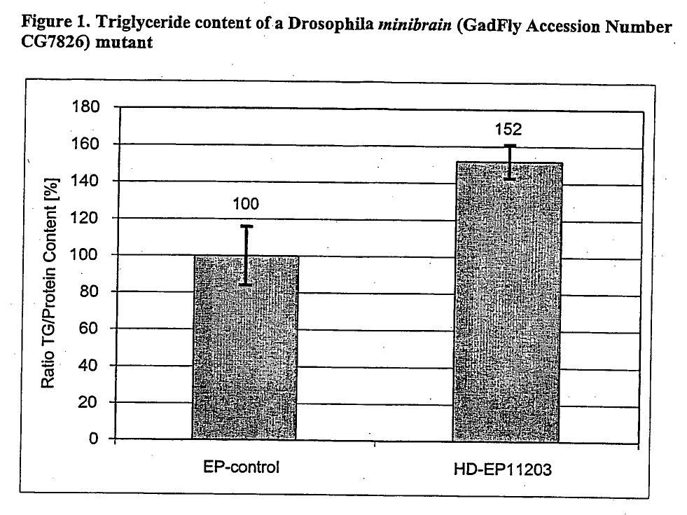

In this invention, the content of triglycerides of a pool of flies with the same genotype was analyzed after prolonged feeding using a triglyceride assay. Male flies hemizygous for the integration of vectors for Drosophila line HD-EP(X)11203 were analyzed in an assay measuring the triglyceride contents of these flies, illustrated in more detail in the Examples section. The results of the triglyceride content analysis are shown in FIG. 1.

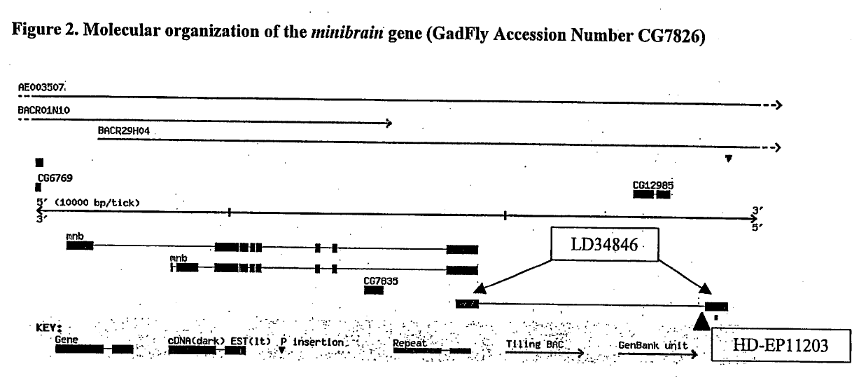

Genomic DNA sequences were isolated that are localized to the EP vector (herein HD-EP(X)11203) integration. Using those isolated genomic sequences public databases like Berkeley Drosophila Genome Project (GadFly; see also FlyBase (1999) Nucleic Acids Research 27: 85-88) were screened thereby identifying the integration site of the vector, and the corresponding gene, described in more detail in the Examples section. The molecular organization of the gene is shown in FIG. 2.

The Drosophila genes and proteins encoded thereby with functions in the regulation of triglyceride metabolism were further analysed in publicly available sequence databases (see Examples for more detail) and mammalian homologs were identified.

The function of the mammalian homologs in energy homeostasis was further validated in this invention by analyzing the expression of the transcripts in different tissues. Expression profiling studies (see Examples for more detail) confirm the particular relevance of the protein(s) of the invention as regulators of energy metabolism in mammals. Further, we show that the proteins of the invention are regulated by fasting and by genetically induced obesity. In this invention, we used mouse models of insulin resistance and/or diabetes, such as mice carrying gene knockouts in the leptin pathway (for example, ob (leptin) or db (leptin receptor) mice) to study the expression of the proteins of the invention. Such mice develop typical symptoms of diabetes, show hepatic lipid accumulation and frequently have increased plasma lipid levels (see Bruning J. C. et al, (1998) Mol. Cell. 2: 559-569).

The invention also encompasses polynucleotide sequences that encode a protein of the invention or a homologous protein. Accordingly, any nucleic acid sequence, which encodes the amino acid sequences of a protein of the invention or a homologous protein, can be used to generate recombinant molecules that express a protein of the invention or a homologous protein. In a particular embodiment, the invention encompasses a nucleic acid encoding Drosophila minibrain, or human minibrain homologs, preferably a human homologous protein as described in Table 1; referred to herein as the proteins of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotide encoding minibrain or a homologous protein, preferably a human homologous protein as described in Table 1, under various conditions of stringency. Hybridization conditions are based on the melting temperature (Tm) of the nucleic acid binding complex or probe, as taught in Wahl G. M. et al., (1987, Methods Enzymol. 152: 399-407) and Kimmel A. R., (1987, Methods Enzymol. 152: 507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivatives thereof such as cyclic peptides, retro-inverso peptides of peptide mimetics having a length of at least 4, preferably at least 6 and up to 50 amino acids.

Also included within the scope of the present invention are alleles of the genes encoding a protein of the invention or a homologous protein. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one or many allelic forms. Common mutational changes, which give rise to alleles, are generally ascribed to natural deletions, additions or substitutions of nucleotides. Each of these types of changes may occur alone or in combination with the others, one or more times in a given sequence.

The nucleic acid sequences encoding the proteins of the invention and homologous proteins may be extended utilizing a partial nucleotide sequence and employing various methods known in the art to detect upstream sequences such as promoters and regulatory elements. For example, one method which may be employed, ‘restriction-site’ PCR, uses universal primers to retrieve unknown sequence adjacent to a known locus (Sarkar G. et al., (1993) PCR Methods Applic. 2: 318-322). Inverse PCR may also be used to amplify or extend sequences using divergent primers based on a known region (Triglia T. et al., (1988) Nucleic Acids Res. 16: 8186). Another method which may be used is capture PCR which involves PCR amplification of DNA fragments adjacent to a known sequence in human and yeast artificial chromosome DNA (Lagerstrom M. et al., (1991) PCR Methods Applic. 1: 111-119). Another method which may be used to retrieve unknown sequences is that of Parker J. D. et al., (1991) Nucleic Acids Res. 19: 3055-3060. Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express a biologically active protein, the nucleotide sequences encoding a protein of the invention or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y. and Ausubel, F. M. et al. (1989) Current Protocols in Molecular Biology, John Wiley & Sons, New York, N.Y.

In a further embodiment of the invention, natural, modified or recombinant nucleic acid sequences encoding the proteins of the invention and homologous proteins may be ligated to a heterologous sequence to encode a fusion protein. Heterologous sequences are preferably located at the N- and/or C-terminus of the fusion protein.

A variety of expression vector/host systems may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences encoding a protein of the invention or a homologous protein in a sample can be detected by DNA-DNA or DNA-RNA hybridization or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of labeled RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of proteins of the invention in a sample can be determined by immunological methods or activity measurement. A variety of protocols for detecting and measuring the expression of proteins, using either polyclonal or monoclonal antibodies specific for the protein or reagents for determining protein activity are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1226).

Suitable reporter molecules or labels, which may be used, include radionuclides, enzymes, fluorescent, chemiluminescent or chromogenic agents as well as substrates, co-factors, inhibitors, magnetic particles, and the like.

Host cells transformed with nucleotide sequences encoding a protein of the invention may be cultured under conditions suitable for the expression and recovery of said protein from cell culture. The protein produced by a recombinant cell may be secreted or contained intracellularly depending on the sequence and/or the vector used. As will be understood by those of skill in the art, expression vectors containing polynucleotides, which encode the protein may be designed to contain signal sequences, which direct secretion of the protein through a prokaryotic or eukaryotic cell membrane. Other recombinant constructions may be used to join sequences encoding the protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the desired protein may be used to facilitate purification.

Diagnostics and Therapeutics

The data disclosed in this invention show that the nucleic acids and proteins of the invention are useful in diagnostic and therapeutic applications implicated, for example but not limited to, in metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, gallstones, or liver fibrosis. Hence, diagnostic and therapeutic uses for the proteins of the invention nucleic acids and proteins of the invention are, for example but not limited to, the following: (i) protein therapy, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention and modulators/effectors thereof are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, metabolic disorders as described above.

The nucleic acids of the invention or fragments thereof, may further be useful in diagnostic applications, wherein the presence or amount of the nucleic acids or the proteins are to be assessed. Further antibodies that bind immunospecifically to the novel substances of the invention may be used in therapeutic or diagnostic methods.

For example, in one aspect, antibodies, which are specific for a protein of the invention or a homologous protein, may be used directly as a modulator/effector, e.g. an antagonist or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Kohler G. and Milstein C., (1975) Nature 256: 495-497; Kozbor D. et al. (1985) J. Immunol. Methods 81: 31-42; Cote R. J. et al., (1983) Proc. Natl. Acad. Sci. 80: 2026-2030; Cole S. P. et al., (1984) Mol. Cell Biochem. 62: 109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. 81: 6851-6855; Neuberger M. S. et al., (1984) Nature 312: 604-608; Takeda S. et al. (1985) Nature 314: 452-454). Alternatively, techniques described for the production of single chain antibodies may be adapted, using methods known in the art, to produce single chain antibodies specific for a protein of the invention or a homologous protein. Antibodies with related specificity, but of distinct idiotypic composition, may be generated by chain shuffling from random combinatorial immunoglobulin libraries (Kang A. S. et al., (1991) Proc. Natl. Acad. Sci. 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. 86: 3833-3837; Winter G. and Milstein C., (1991) Nature 349: 293-299).

Antibody fragments which contain specific binding sites for the proteins may also be generated. For example, such fragments include, but are not limited to, the F(ab′)2 fragments which can be produced by Pepsin digestion of the antibody molecule and the Fab fragments which can be generated by reducing the disulfide bridges of F(ab′)2 fragments. Alternatively, Fab expression libraries may be constructed to allow rapid and easy identification of monoclonal Fab fragments with the desired specificity (Huse W. D. et al., (1989) Science 246: 1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering protein epitopes are preferred, but a competitive binding assay may also be employed (Maddox, D. E. et al., supra).

In another embodiment of the invention, the polynucleotides of the invention or fragments thereof or nucleic acid effector molecules such as antisense molecules or ribozymes may be used for therapeutic purposes. In one aspect, antisense molecules may be used in situations in which it would be desirable to block the transcription of the mRNA. In particular, cells may be transformed with sequences complementary to polynucleotides encoding a protein of the invention or a homologous protein. Thus, antisense molecules may be used to modulate protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding a protein of the invention or a homologous protein. These techniques are described both in Sambrook et al. (supra) and in Ausubel et al. (supra). Genes encoding a protein of the invention or a homologous protein can be turned off by transforming a cell or tissue with expression vectors, which express high levels of polynucleotides that encode a protein of the invention or a homologous protein or functional fragments thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA or PNA, to the control regions of the genes encoding a protein of the invention or a homologous protein, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee J. E. et al., (1994) Gene 149: 109-114; Huber B. E. and Carr B. I., (1994) Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding a protein of the invention or a homologous protein. Specific ribozyme cleavage sites within any potential RNA target are initially identified by scanning the target molecule for ribozyme cleavage sites which include the following sequences: GUA, GUU, and GUC Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of the nucleic acids or/and proteins of the invention or/and homologous nucleic acids or/and proteins, antibodies to a protein of the invention or/and a homologous protein, mimetics, agonists, antagonists or/and inhibitors of a protein of the invention or/and a homologous protein or/and nucleic acid sequence. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes. The pharmaceutical composition may be provided as a salt and can be formed with many acids. After pharmaceutical compositions have been prepared, they can be placed in an appropriate container and labeled for treatment of an indicated condition. For administration of proteins, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example the nucleic acids or the proteins of the invention or homologous proteins or nucleic acids or fragments thereof, antibodies of a protein of the invention or a homologous protein, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or underexpression of a protein of the invention or a homologous protein or in assays to monitor patients being treated with the proteins of the invention or homologous proteins, or effectors thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometry, means. Quantities of protein expressed in control and disease, samples from biopsied tissues are compared with the standard values. Deviation between standard and subject values establishes the parameters for diagnosing disease.

In another embodiment of the invention, the polynucleotides specific for a protein of the invention or a homologous protein may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the proteins of the invention and homologous proteins or closely related molecules, may be used to identify nucleic acid sequences which encode the respective protein. The hybridization probes of the subject invention may be DNA or RNA and derived from the nucleotide sequence of the polynucleotide encoding the proteins of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for the proteins of the invention and homologous nucleic acids may be used for the diagnosis of conditions or diseases, which are associated with the expression of the proteins. Examples of such conditions or diseases include, but are not limited to, metabolic diseases and disorders, including obesity and diabetes. Polynucleotide sequences specific for the proteins of the invention and homologous proteins may also be used to monitor the progress of patients receiving treatment for metabolic diseases and disorders, including obesity and diabetes. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression.

In a particular aspect, the nucleotide sequences specific for the proteins of the invention and homologous nucleic acids may be useful in assays that detect activation or induction of various metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, gallstones, or liver fibrosis. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable have hybridized with nucleotide sequences in the sample, and the presence of altered levels of nucleotide sequences encoding the proteins of the invention and homologous proteins in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the proteins of the invention and homologous proteins, a normal or standard profile for expression is established. This may be accomplished by combining body fluids or cell extracts taken from normal subjects, either animal or human, with a sequence or a fragment thereof, which is specific for the nucleic acids encoding the proteins of the invention and homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as described above the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders. Additional diagnostic uses for oligonucleotides designed from the sequences encoding the proteins of the invention and homologous proteins may involve the use of PCR. Such oligomers may be chemically synthesized, generated enzymatically or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers or even a degenerate pool of oligomers may be employed under less stringent conditions for detection and/or quantification of closely related DNA or RNA sequences.

Methods which may also be used to quantitate the expression of a protein of the invention or a homologous protein include radiolabeling or biotinylating nucleotides, coamplification of a control nucleic acid, and standard curves onto which the experimental results are interpolated (Melby P. C. et al., (1993) J. Immunol. Methods, 159: 235-244; Duplaa C. et al., (1993) Anal. Biochem. 212: 229-236). The speed of quantification of multiple samples may be accelerated by running the assay in an ELISA format where the oligomer of interest is presented in various dilutions and a spectrophotometric or calorimetric response gives rapid quantification.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.). The results may be correlated with other physical chromosome mapping techniques and genetic map data. Examples of genetic map data can be found in the 1994 Genome Issue of Science (265:1981f). Correlation between the location of the gene encoding the proteins of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier, or affected individuals. In situ hybridisation of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localised by genetic linkage to a particular genomic region, for example, AT to 11q22-23 (Gatti R. A. et al., (1988) Nature 336: 577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, the proteins of the invention, their catalytic or immunogenic fragments or oligopeptides thereof, an in vitro model, a genetically altered cell or animal, can be used for screening libraries of compounds, e.g. peptides or low molecular weight organic compounds, in any of a variety of drug screening techniques. One can identify modulators/effectors, e.g. receptors, enzymes, proteins, ligands, or substrates that bind to, modulate or mimic the action of one or more of the proteins of the invention. The protein or functional fragment thereof employed in such screening may be free in solution, affixed to a solid support, borne on a cell surface, or located intracellularly. The formation of binding complexes, between the protein and the agent tested, may be measured. Agents could also, either directly or indirectly, influence the activity of the proteins of the invention.

In vivo, the enzymatic kinase activity of the unmodified polypeptides of minibrain homologous kinase towards a substrate can be measured. Activation of the kinase may be induced in the natural context by extracellular or intracellular stimuli, such as signaling molecules or environmental influences. One may generate a system containing minibrain homologous kinase may it be an organism, a tissue, a culture of cells or cell-free environment, by exogenously applying this stimulus or by mimicking this stimulus by a variety of the techniques, some of them described further below. A system containing activated minibrain homologous kinase may be produced (i) for the purpose of diagnosis, study, prevention, and treatment of diseases and disorders related to body-weight regulation and thermogenesis, for example, but not limited to, metabolic diseases, (ii) for the purpose of identifying or validating therapeutic candidate agents, pharmaceuticals or drugs that influence the genes of the invention or their encoded polypeptides, (iii) for the purpose of generating cell lysates containing activated polypeptides encoded by the genes of the invention, (iv) for the purpose of isolating from this source activated polypeptides encoded by the genes of the invention.

In one embodiment of the invention, one may produce activated minibrain homologous kinase independent of the natural stimuli for the above said purposes by, for example, but not limited to, (i) an agent that mimics the natural stimulus; (ii) an agent, that acts downstream of the natural stimulus, such as activators of minibrain homologous kinase, constitutive active alleles of minibrain homologous kinase itself as they are described or may be developed; (iii) by introduction of single or multiple amino acid substitutions, deletions or insertions within the sequence of minibrain homologous kinase to yield constitutive active forms; (iv) by the use of isolated fragments of minibrain homologous kinase. In addition, one may generate enzymatically active minibrain homologous kinase in an ectopic system, prokaryotic or eukaryotic, in vivo or in vitro, by co-transfering to this system the activating components.

In addition activity of minibrain homologous kinase against its physiological substrate(s) or derivatives thereof could be measured in cell-based assays. Agents may also interfere with posttranslational modifications of the proteins of the invention, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, ion channels, uncoupling proteins, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from a protein of the invention to the interacting protein (or vice versa) could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known In the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interactions are minibrain homologous kinases.

Assays for determining enzymatic, carrier, or ion channel activity of the proteins of the invention are well known in the art. Well known in the art are also a variety of assay formats to measure receptor-ligand binding.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Candidate agents may also be found in kinase assays where a kinase substrate such as a protein, a peptide, a lipid, or an organic compound, which may or may not include modifications as further described below, or others are phosphorylated by the proteins or protein fragments of the invention. The kinase can be a protein of the invention (e.g. minibrain homologous kinase) or a kinase which is influenced in its activity by a protein of the invention. A therapeutic candidate agent may be identified by its ability to increase or decrease the enzymatic activity of the proteins of the invention. The kinase activity may be detected by change of the chemical, physical or immunological properties of the substrate due to phosphorylation. One example could be the transfer of radioisotopically labelled phosphate groups from an appropriate donor molecule to the kinase substrate catalyzed by the polypeptides of the invention. The phosphorylation of the substrate may be followed by detection of the substrates autoradiography with techniques well known in the art.

Yet in another example, the change of mass of the substrate due to its phosphorylation may be detected by mass spectrometry techniques. One could also detect the phosphorylation status of a substrate with an analyte discriminating between the phosphorylated and unphosphorylated status of the substrate. Such an analyte may act by having different affinities for the phosphorylated and unphosphorylated forms of the substrate or by having specific affinity for phosphate groups. Such an analyte could be, but is not limited to, an antibody or antibody derivative, a recombinant antibody-like structure, a protein, a nucleic acid, a molecule containing a complexed metal ion, an anion exchange chromatography matrix, an affinity chromatography matrix or any other molecule with phosphorylation dependend selectivity towards the substrate.

Such an analyte could be employed to detect the kinase substrate, which is immobilized on a solid support during or after an enzymatic reaction. If the analyte is an antibody, its binding to the substrate could be detected by a variety of techniques as they are described in Harlow and Lane, 1998, Antibodies, CSH Lab Press, NY. If the analyte molecule is not an antibody, it may be detected by virtue of its chemical, physical or immunological properties, being endogenously associated with it or engineered to it.

Yet in another example the kinase substrate may have features, designed or endogenous, to facilitate its binding or detection in order to generate a signal that is suitable for the analysis of the substrates phosphorylation status. These features may be, but are not limited to, a biotin molecule or derivative thereof, a glutathione-S-transferase moiety, a moiety of six or more consecutive histidine residues, an amino acid sequence or hapten to function as an epitope tag, a fluorochrome, an enzyme or enzyme fragment. The kinase substrate may be linked to these or other features with a molecular spacer arm to avoid steric hindrance.

In one example, the kinase substrate may be labelled with a fluorochrome. The binding of the analyte to the labelled substrate in solution may be followed by the technique of fluorescence polarization as it is described in the literature (see, for example, Deshpande S. et al., (1999) Prog. Biomed. Optics (SPIE) 3603: 261; Parker G. J. et al., (2000) J. Biomol. Screen. 5: 77-88; Wu P. et al., (1997) Anal. Biochem. 249: 29-36). In a variation of this example, a fluorescent tracer molecule may compete with the substrate for the analyte to detect kinase activity by a technique which is known to those skilled in the art as indirect fluorescence polarization.

Another technique for drug screening, which may be used, provides for high throughput screening of compounds having suitable binding affinity to the protein of interest as described in published PCT application WO84/03564. In this method, as applied to the proteins of the invention large numbers of different small test compounds are synthesised on a solid substrate, such as plastic pins or some other surface. The test compounds are reacted with a protein of the invention, or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilise it on a solid support.

In another embodiment, one may use competitive drug screening assays in which neutralising antibodies capable of binding minibrain homologous kinase specifically compete with a test compound for binding minibrain homologous kinase. In this manner, the antibodies can be used to detect the presence of any peptide, which shares one or more antigenic determinants with minibrain homologous kinase.

The nucleic acids encoding a protein of the invention can be used to generate transgenic animals or site-specific gene modifications in cell lines. These transgenic non-human animals are useful in the study of the function and regulation of said protein in vivo. Transgenic animals, particularly mammalian transgenic animals, can serve as a model system for the investigation of many developmental and cellular processes common to humans. A variety of non-human models of metabolic disorders can be used to test modulators/effectors of the proteins of the invention. Misexpression (for example, overexpression or lack of expression) of a protein of the invention, particular feeding conditions, and/or administration of biologically active compounds can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning J. C. et al, (1998) Mol. Cell. 2: 559-569). Susceptible wild type mice (for example C57BI/6) show similiar symptoms if fed a high fat diet. In addition to testing the expression of the proteins of the invention in such mouse strains (see Examples section), these mice could be used to test whether administration of a candidate modulator/effector alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in non-human embryonic stem cells, where the normal locus of the gene encoding a protein of the invention is altered. Alternatively, a nucleic acid construct encoding a protein of the invention is injected into oocytes and is randomly integrated into the genome. Vectors for stable integration include plasmids, retroviruses and other animal viruses, yeast artificial chromosomes (YACs), and the like. The modified cells or animal are useful in the study of the function and regulation of the proteins of the invention. For example, a series of small deletions and/or substitutions may be made in the gene that encodes a protein of the invention to determine the role of particular domains of the protein, functions in pancreatic differentiation, etc.

Furthermore, variants of the genes of the invention like specific constructs of interest include anti-sense molecules, which will block the expression of the proteins of the invention, or expression of dominant negative mutations. A detectable marker, such as for example lac-Z or luciferase may be introduced in the locus of a gene of the invention, where up regulation of expression of the genes of the invention will result in an easily detected change in phenotype.

One may also provide for expression of the genes of the invention or variants thereof in cells or tissues where it is not normally expressed or at abnormal times of development. In addition, by providing expression of the proteins of the invention in cells in which they are not normally produced, one can induce changes in cell behavior.

DNA constructs for homologous recombination will comprise at least portions of the genes of the invention with the desired genetic modification, and will include regions of homology to the target locus. DNA constructs for random integration do not need to contain regions of homology to mediate recombination. Conveniently, markers for positive and negative selection are included. DNA constructs for random integration will consist of the nucleic acids encoding the proteins of the invention, a regulatory element (promoter), an intron and a poly-adenylation signal. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or embryonic cells or somatic pluripotent stem cells have been transfected, they may be used to produce transgenic animals. After transfection, the cells are plated onto a feeder layer in an appropriate medium. Cells containing the construct may be selected by employing a selective medium. After sufficient time for colonies to grow, they are picked and analyzed for the occurrence of homologous recombination or integration of the construct. Those colonies that are positive may then be used for embryo transfection and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a nucleic acid molecule coding for a protein of the invention or a functional fragment thereof;
(b) a protein of the invention or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another modulator/effector of the nucleic acid of (a) or the polypeptide of (b), (e), or (f) and
(h) an anti-sense oligonucleotide of the nucleic acid of (a).

The kit may be used for diagnostic or therapeutic purposes or for screening applications as described above. The kit may further contain user instructions.

The Figures show:

FIG. 1 shows the triglyceride content of a Drosophila minibrain (GadFly Accession Number CG7826) mutant. Shown is the change of triglyceride content of HD-EP(X)11203 flies caused by hemizygous viable integration of the P-vector into an intron (217 base pairs 3′ of the first exon of LD34846) of the annotated cDNA (referred to as ‘HD-EP11203’, column 2) in comparison to controls containing all flies of the EP collection (referred to as ‘EP-control’, column 1).

FIG. 2 shows the molecular organization of the mutated minibrain gene locus.

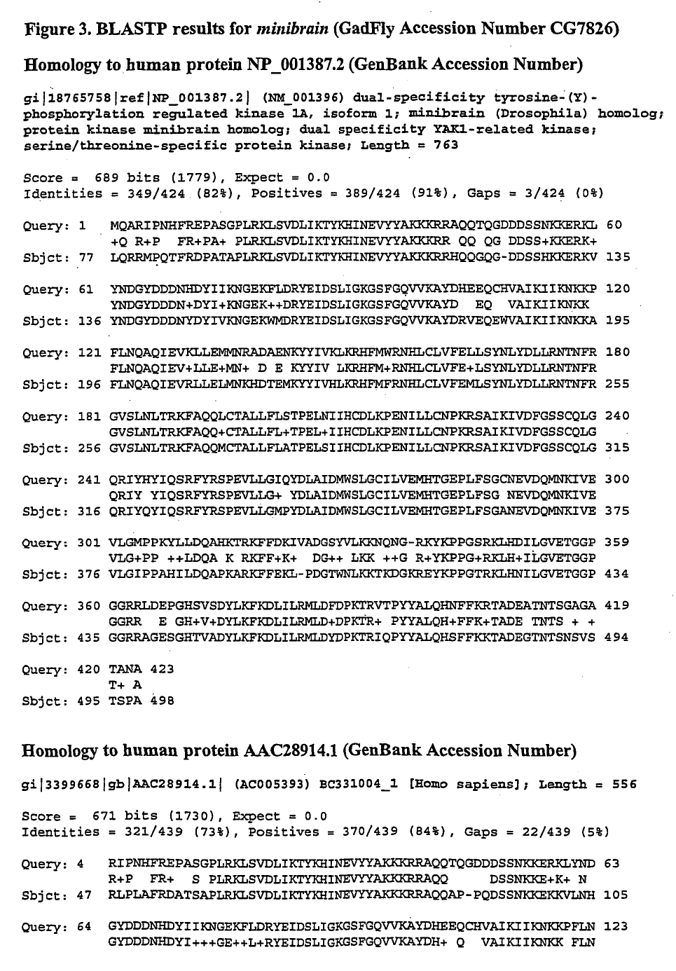

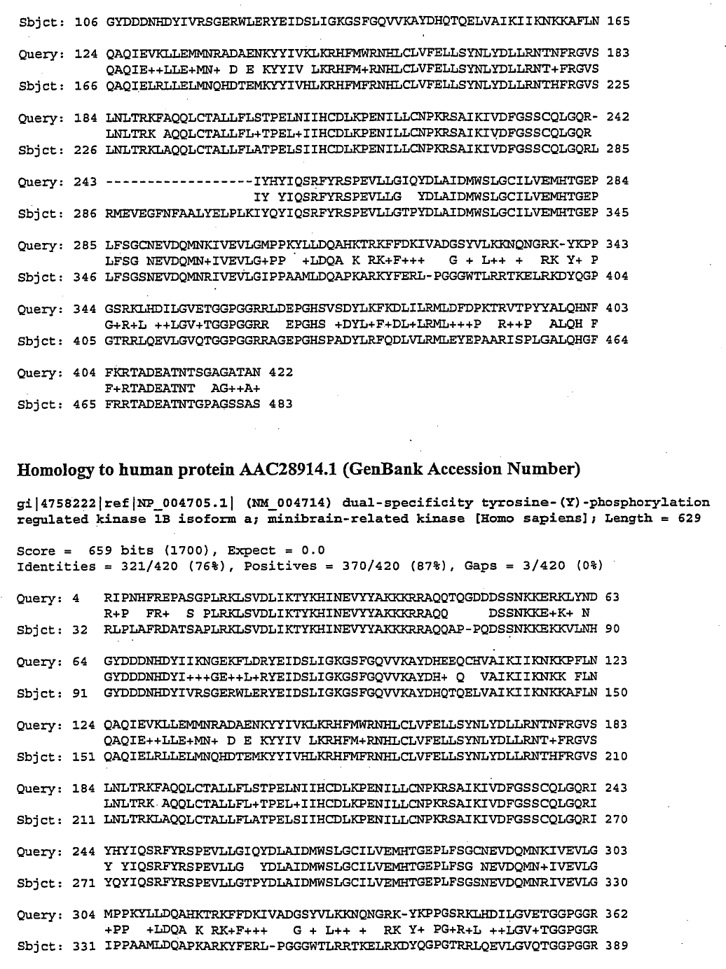

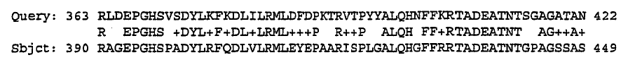

FIG. 3 shows the BLASTP search results for the minibrain gene product (Query) with the three best human homologous matches (Sbjct).

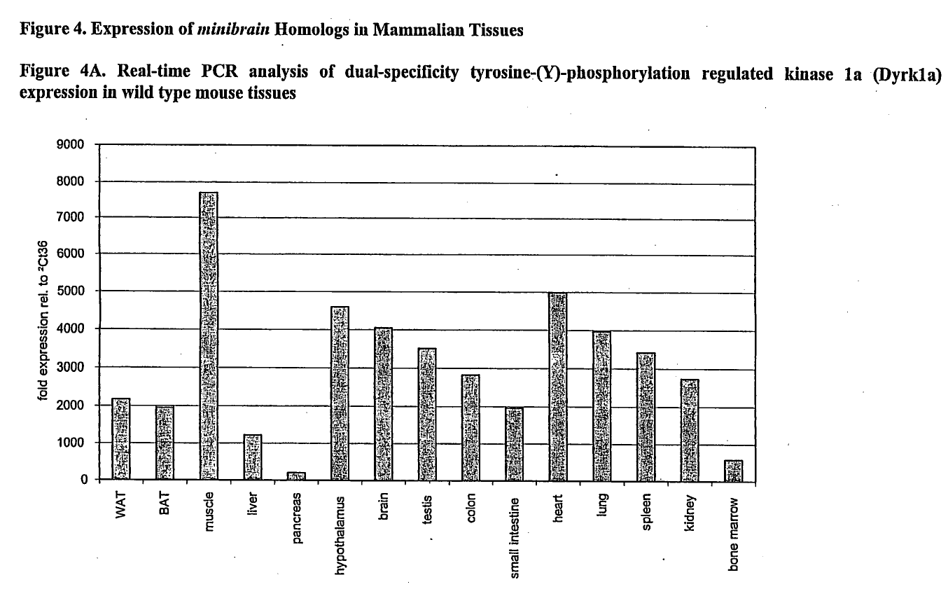

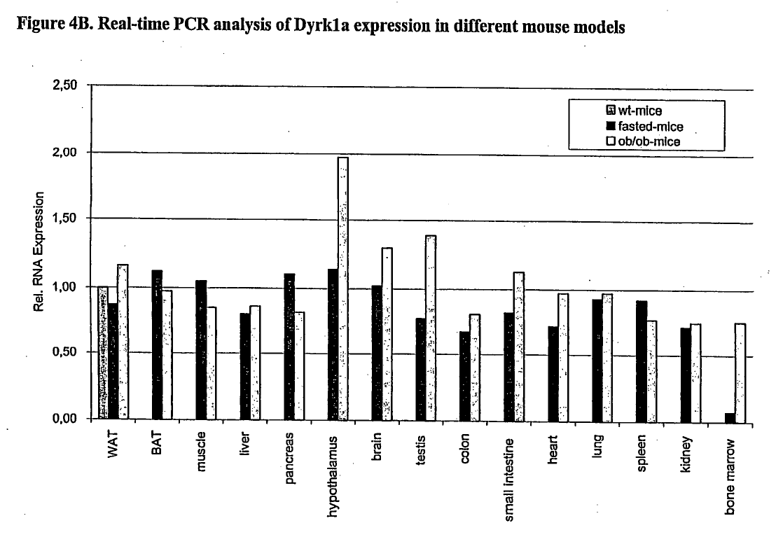

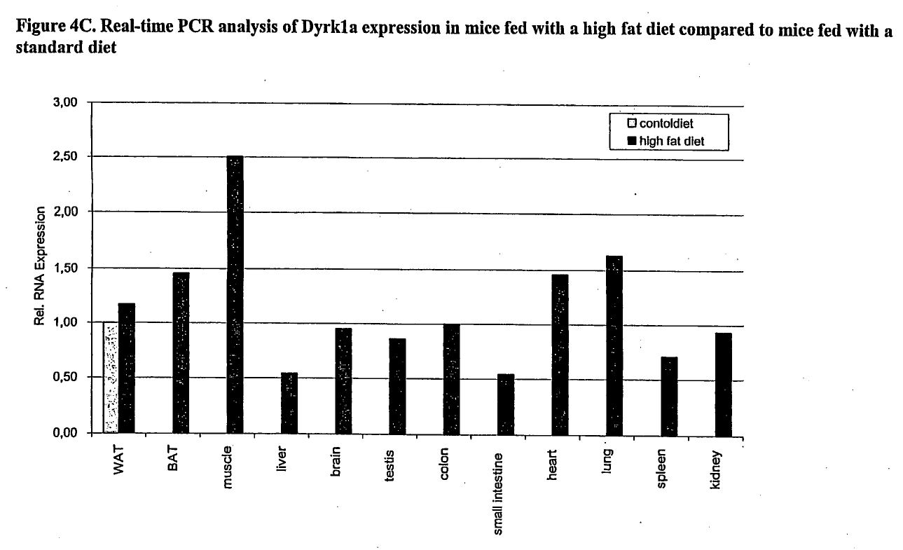

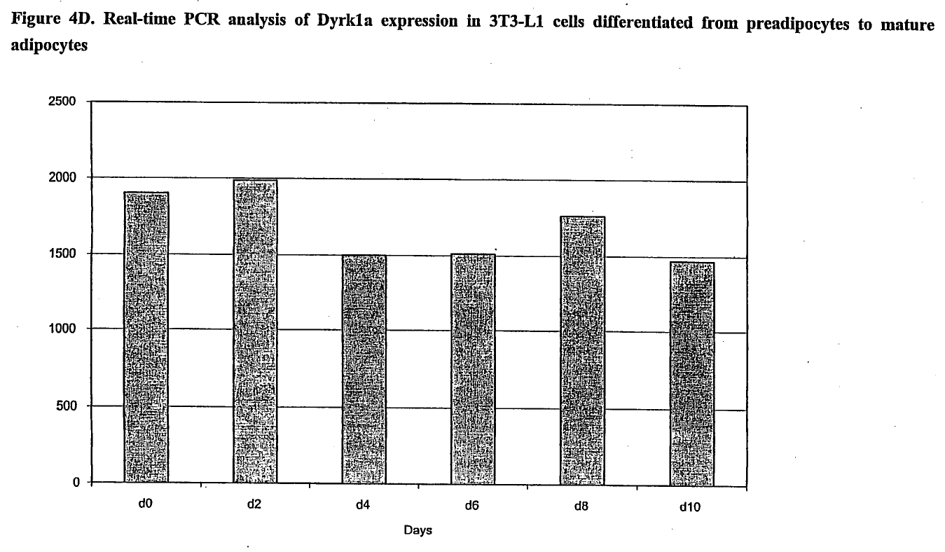

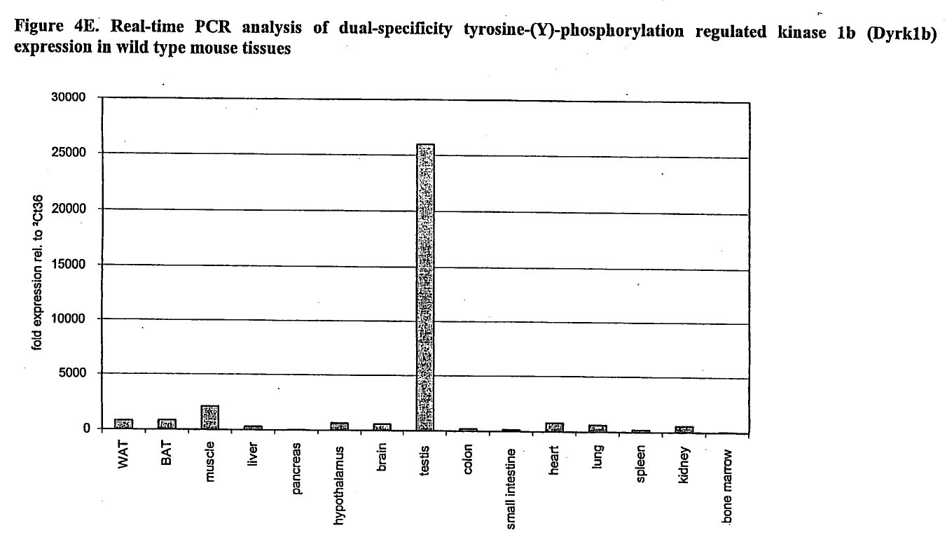

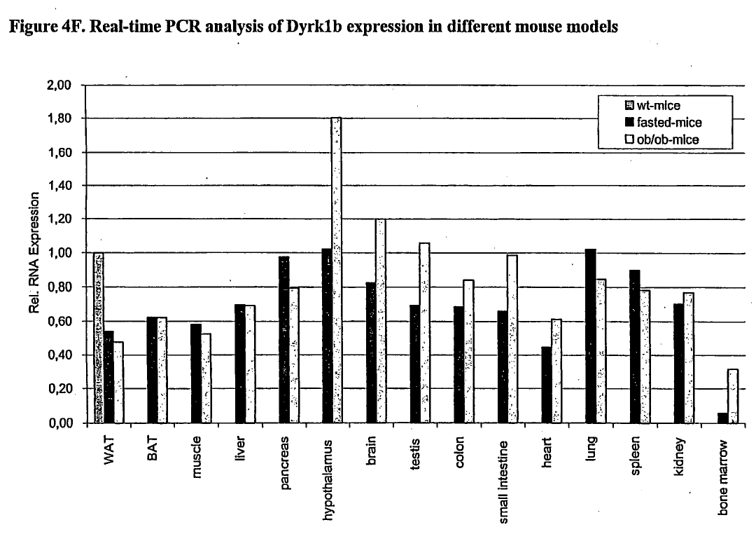

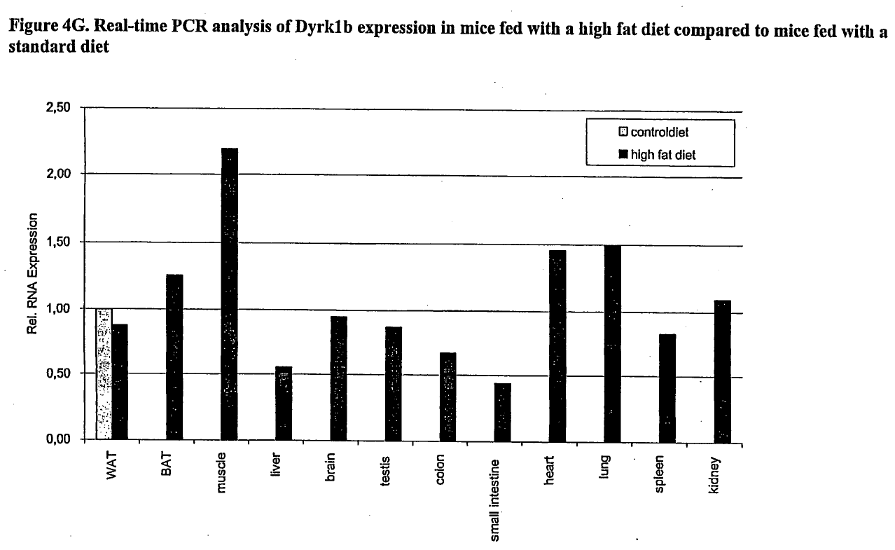

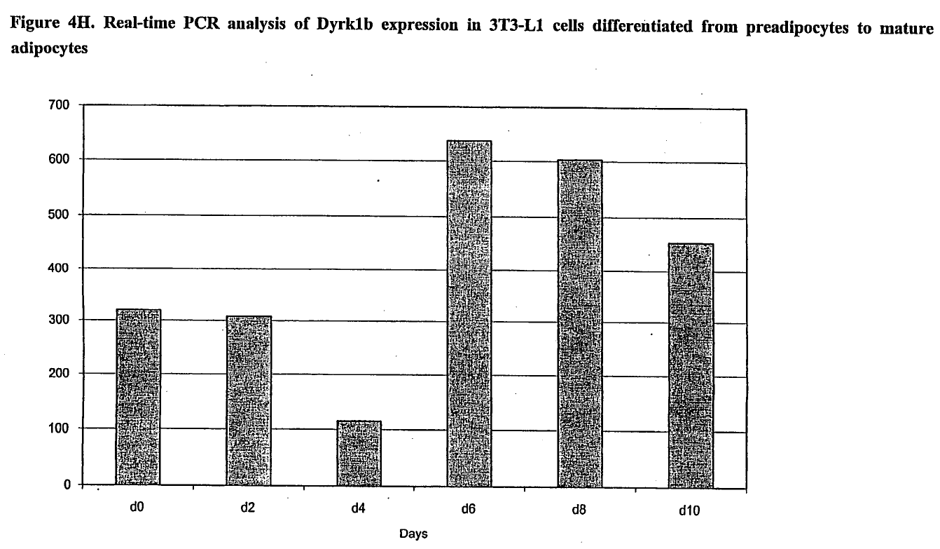

FIG. 4 shows the expression of two minibrain homologs in mammalian (mouse) tissues.

FIG. 4A shows the real-time PCR analysis of dual-specificity tyrosine-(Y)-phosphorylation regulated kinase 1a (Dyrk1a) expression in wild-type mouse tissues.

FIG. 4B shows the real-time PCR analysis of Dyrk1a expression in different mouse models.

FIG. 4C shows the real-time PCR analysis of Dyrk1a expression in mice fed with a high fat diet compared to mice fed with a standard diet.

FIG. 4D shows the real-time PCR analysis of Dyrk1a expression during the differentiation of 3T3-L1 cells from preadipocytes to mature adipocytes.

FIG. 4E shows the real-time PCR analysis of dual-specificity tyrosine-(Y)-phosphorylation regulated kinase 1b (Dyrk1b) expression in wild-type mouse tissues.

FIG. 4F shows the real-time PCR analysis of Dyrk1b expression in different mouse models.

FIG. 4G shows the real-time PCR analysis of Dyrk1b expression in mice fed with a high fat diet compared to mice fed with a standard diet.

FIG. 4H shows the real-time PCR analysis of Dyrk1b expression during the differentiation of 3T3-L1 cells from preadipocytes to mature adipocytes.

The examples illustrate the invention:

### EXAMPLE 1

### Measurement of Triglyceride Content in Drosophila

Mutant flies are obtained from a fly mutation stock collection. The flies are grown under standard conditions known to those skilled in the art. In the course of the experiment, additional feedings with bakers yeast (Saccharomyces cerevisiae) are provided for the EP-line HD-EP(X)11203. The average change of triglyceride content of Drosophila containing the EP-vector as hemizygous viable integration was investigated in comparison to control flies (see FIG. 1). For determination of triglyceride content, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference the protein content of the same extract was measured using BIO-RAD DC Protein Assay according to the manufacturer's protocol. These assays were repeated several times.

The average triglyceride level of all flies of the EP collection (referred to as ‘EP-control’) is shown as 100% in the first column in FIG. 1. Standard deviations of the measurements are shown as thin bars.

HD-EP(X)11203 hemizygous flies show constantly a higher triglyceride content than the controls (column 2 in FIG. 1, ‘HD-EP11203’). Therefore, the loss of gene activity is responsible for changes in the metabolism of the energy storage triglycerides.

### EXAMPLE 2

### Identification of Drosophila Genes Associated with Triglyceride Metabolism

Nucleic acids encoding the proteins of the present invention were identified using a plasmid-rescue technique. Genomic DNA sequences were isolated that are localized directly adjacent to the EP vector (herein HD-EP(X)11203) integration. Using those isolated genomic sequences public databases like Berkeley Drosophila Genome Project (GadFly) were screened thereby confirming the hemizygous viable integration site of the HD-EP(X)11203 into an intron of LD34846 (217 base pairs 3′ of the first exon of LD34846), a transcribed sequence overlapping with the sequence of a Drosophila gene in sense orientation, identified as minibrain (GadFly Accession Number CG7826). FIG. 2 shows the molecular organization of this gene locus. The chromosomal localization site of integration of the vector of HD-EP(X)11203 is at gene locus X, 16E4-F1. In FIG. 2, genomic DNA sequence is represented by the assembly as a black scaled double-headed arrow in the middle of the figure that includes the integration site of HD-EP(X)11203. Ticks represent the length in basepairs of the genomic DNA (10000 base pairs per tick). The grey arrows in the upper part of the figures represent BAC clones, the black arrow in the topmost part of the figure represents the section of the chromosome. The insertion site of the P-element in the Drosophila line is shown as triangle and is labeled. The cDNA sequences of the predicted genes (as predicted by the Berkeley Drosophila Genome Project, GadFly and by Magpie) are shown as dark grey bars (exons), linked by dark grey lines (introns), and are labeled (see also key at the bottom of the figures). The predicted cDNAs of the minibrain gene (referred to as mnb) is shown in the lower half of the figure. Additionally the transcribed sequence of LD34846, which is overlapping with the minibrain gene, is shown in the lower half of the figure and is labeled. Therefore, expression of the cDNA encoding minibrain could be affected by integration of vectors of line HD-EP(X)11203, leading to a change in the amount of energy storage triglycerides.

### EXAMPLE 3

### Identification of Mammalian Homologous Genes and Proteins

The Drosophila genes and proteins encoded thereby with functions in the regulation of triglyceride metabolism were further analysed using the BLAST algorithm searching in publicly available sequence databases and mammalian homologs were identified (see Table 1 and FIG. 3).

The term “polynucleotide comprising the nucleotide sequence as shown in GenBank Accession number” relates to the expressible gene of the nucleotide sequences deposited under the corresponding GenBank Accession number. The term “GenBank Accession number” relates to NCBI GenBank database entries (Ref.: Benson D. A. et al., (2000) Nucleic Acids Res. 28: 15-18). Sequences homologous to Drosphila minibrain were identified using the publicly available program BLASTP 2.2.3 of the non-redundant protein data base of the National Center for Biotechnology Information (NCBI) (see, Altschul S. F. et al., (1997) Nucleic Acids Res. 25: 3389-3402).

TABLE 1
Human homologs of the Drosophila (Dm) genes

| Dm gene | Homo sapiens homologous genes and proteins | | |
|---|---|---|---|
| Acc. No. | Accession Number | | |
| Name | cDNA | Protein | Name |
| CG7826 | NM_001396 | NP_001387 | dual-specificity tyrosine-(Y)- |
| minibrain | | | phosphorylation regulated kinase |
| | | | 1A isoform 1 |
| | NM_130436 | NP_569120 | dual-specificity tyrosine-(Y)- |
| | | | phosphorylation regulated kinase |
| | | | 1A isoform 2 |
| | NM_101395 | NP_567824 | dual-specificity tyrosine-(Y)- |
| | | | phosphorylation regulated kinase |
| | | | 1A isoform 3 |
| | NM_130437 | NP_569121 | dual-specificity tyrosine-(Y)- |
| | | | phosphorylation regulated kinase |
| | | | 1A isoform 4 |
| | NM_130438 | NP_569122 | dual-specificity tyrosine-(Y)- |
| | | | phosphorylation regulated kinase |
| | | | 1A isoform 5 |
| | NM_004714 | NP_004705 | dual-specificity tyrosine-(Y)- |
| | | | phosphorylation regulated kinase |
| | | | 1B isoform a |
| | NM_006483 | NP_006474 | dual-specificity tyrosine-(Y)- |
| | | | phosphorylation regulated kinase |
| | | | 1B isoform b |
| | NM_006484 | NP_006475 | dual-specificity tyrosine-(Y)- |
| | | | phosphorylation regulated kinase |
| | | | 1B isoform c |
| | AC005393 | AAC28914 | chromosome 19, CIT-HSP BAC |
| | | | 470n8; BC331004_1 |

Minibrain homologous proteins and nucleic acid molecules coding therefore are obtainable from insect or vertebrate species, e.g. mammals or birds. Particularly preferred are nucleic acids as described in Table 1.

As shown in FIG. 3, the gene product of Drosophila minibrain (GadFly Accession Number CG7826, GenBank Accession Number NM_{—}078668 for the cDNA, AAF48777 for the protein) is 91% homologous to human dual-specificity tyrosine-(Y)-phosphorylation regulated kinase 1A, isoform 1 (GenBank Accession Number NP_{—}001387 for the protein, NM_{—}001396 for the cDNA; also disclosed in U.S. Pat. No. 5,981,250, U.S. Pat. No. 6,107,074, and U.S. Pat. No. 6,251,664 as well as in WO 01/88188 and WO 01/194629 (98% identity)), 84% homologous to human BC331004_{—}1 protein (GenBank Accession Number AAC28914.1 for the protein, AC005393 for the cDNA), and 87% homologous to human dual-specificity tyrosine-(Y)-phosphorylation regulated kinase 1B isoform a (GenBank Accession Number NP 004705.1 for the protein, NM_{—}004714 for the cDNA). Drosophila minibrain also shows homology on protein level to further isoforms of human DYRK1A (GenBank Accession Numbers NM_{—}01395, NM_{—}130436, NM_{—}130437, and NM_{—}130438 for the cDNAs, NP_{—}567824, NP_{—}569120, NP_{—}569121, NP_{—}569122, BAA33975, BAA33976, BAA33977, AAD31169, CAA05059, CAA05060, BAA12866, BAA13110, AAB18639, AAC50939, CAA80910 for the proteins). Drosophila minibrain also shows homology on protein level to further isoforms of human DYRK1B (GenBank Accession Numbers NM_{—}004714, NM_{—}006483, NM_{—}006484 for the cDNAs, NP_{—}004705, NP_{—}006474, NP_{—}006475, AAF15893, AAH18751, AAH25291, CAA76989, CAA76990, CAA76991 for the proteins). Drosophila minibrain also shows homology on protein level to further human protein (GenBank Accession Number AAC28914). Drosophila minibrain also shows homologyon protein level to murine Dyrk1a (GenBank Accession Number XM_{—}128336 for the cDNA) and to murine Dyrk1b (GenBank Accession Number NM 010092 for the cDNA).

### EXAMPLE 4

### Expression of the Polypeptides in Mammalian (Mouse) Tissues

To analyse the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferably 22° C.), 40 percent humidity and a light/dark cycle of preferrably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum (see, for example, Schnetzler B. et al., 1993, J Clin Invest 92: 272-280, Mizuno T. M. et al., 1996, Proc Natl Acad Sci USA 93: 3434-3438). In a further experiment wild-type (wt) mice were fed a control diet (preferably Altromin C1057 mod control, 4.5% crude fat) or high fat diet (preferably Altromin C1057mod. high fat, 23.5% crude fat). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of mammalian cell culture cells for the conversion of pre-adipocytes to adipocytes, mammalian fibroblast (3T3-L1) cells (e.g., Green H. and Kehinde O., 1974, Cell 1: 113-116) were obtained from the American Tissue Culture Collection (ATCC, Hanassas, VA, USA; ATCC-CL 173). 3T3-L1 cells were maintained as fibroblasts and differentiated into adipocytes as described in the prior art (e.g., Qiu Z. et al., 2001, J. Biol. Chem. 276: 11988-11995; Slieker L. J. et al., 1998, BBRC 251: 225-229). In brief, cells were plated in DMEM/10% FCS (Invitrogen, Karlsruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/HamF12 (3:1; Invitrogen), fetuin (300 μg/ml; Sigma, Munich, Germany), transferrin (2 μg/ml; Sigma), pantothenate (17 μM; Sigma), biotin (1 μM; Sigma), and EGF (0.8 nM; Hoffmann-La Roche, Basel, Switzerland). Differentiation was induced by adding dexamethasone (DEX; 1 μM; Sigma), 3-methyl-isobutyl-1-methylxanthine (MIX; 0.5 mM; Sigma), and bovine insulin (5 μg/ml; Invitrogen). Four days after confluence (d4), cells were kept in SF medium, containing bovine insulin (5 μg/ml) until differentiation was completed. At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

RNA was isolated from tissues and cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH-Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis was performed preferably using the following primer/probe pairs:

For the amplification of mouse dual-specificity tyrosine-(Y)-phosphorylation regulated kinase 1a (Dyrk1a) sequence (GenBank Accession Number XM_{—}128336):

Mouse Dyrk1a forward primer:

| | |
|---|---|
| (SEQ ID NO: 1) | 5′- TGC GAT GGA GCA GTC TCA GT -3′; |

mouse Dyrk1a reverse primer:

| | |
|---|---|
| (SEQ ID NO: 2) | 5′- GAG GAT CCA CCT GAG CTG GA -3′; |

mouse Dyrk1a Taqman probe:

| | |
|---|---|
| (SEQ ID NO: 3) | (5/6-FAM)- TTC AGG CAC CAC CTC CAG |
| | CAC CTC -(5/6-TAMRA). |

For the amplification of mouse dual-specificity tyrosine-(Y)-phosphorylation regulated kinase 1b (Dyrk1b) sequence (GenBank Accession Number NM_{—}010092):

Mouse Dyrk1b forward primer:

| | |
|---|---|
| (SEQ ID NO: 4) | 5′- TGG GCT GCA TCC TCG TG -3′; |

mouse Dyrk1b reverse primer:

| | |
|---|---|
| (SEQ ID NO: 5) | 5′- CAT CTG GTC CAC CTC ATT AGA |
| | GC -3′; |

mouse Dyrk1b Taqman probe:

| | |
|---|---|
| (SEQ ID NO: 6) | (5/6-FAM)- AGA TGC ACA CCG GAG AGC |
| | CCC TCT T -(5/6-TAMRA). |

In FIG. 4 the relative RNA-expression is shown on the Y-axis. In FIG. 4A-C and 4E-G, the tissues tested are given on the X-axis. “WAT” refers to white adipose tissue, “BAT” refers to brown adipose tissue. In FIGS. 4D and 4H, the X-axis represents the time axis. “d0” refers to day 0 (start of the experiment), “d2”-“d10” refers to day 2-day 10 of adipocyte differentiation.

The function of the proteins of the invention in metabolism was further validated by analyzing the expression of the transcripts in different tissues and by analyzing the role in adipocyte differentiation. Expression profiling studies confirm the particular relevance of Dyrk1a and Dyrk1b as regulators of energy metabolism in mammals.

In one embodiment of this invention, mouse models of insulin resistance or/and diabetes were used, such as mice carrying gene knockouts in the leptin pathway (for example, ob/ob (leptin) or db/db (leptin receptor/ligand) mice) to study the expression of the proteins of the invention. Such mice develop typical symptoms of diabetes, show hepatic lipid accumulation and frequently have increased plasma lipid levels (see Bruning J. C. et al, (1998) Mol. Cell. 2: 559-569).

In a further embodiment of the invention, expression of the mRNAs encoding the proteins of the invention was also examined in susceptible wild type mice (for example, C57BI/6) that show symptoms of diabetes, lipid accumulation, and high plasma lipid levels, if fed a high fat diet.

Taqman analysis revealed that Dyrk1a is expressed in several mammalian tissues, showing highest level of expression in muscle, and higher levels in further tissues, e.g. white adipose tissue (WAT), brown adipose tissue (BAT), hypothalamus, brain, testis, colon, small intestine, heart, lung, spleen, and kidney. Furthermore Dyrk1a is expressed on lower but still robust levels in liver, pancreas, and bone marrow of wild type mice as depicted in FIG. 4A. We found, for example, that the expression of Dyrk1a is slightly up regulated in the hypothalamus of genetically induced obese mice (ob/ob) compared to wild type mice. Furthermore Dyrk1a is down regulated in bone marrow of fasted mice compared to wild type mice (see FIG. 4B). In wild type mice fed a high fat diet, the expression of Dyrk1a is up regulated in muscle and slightly down regulated in liver and small intestine, as depicted in FIG. 4C. We show in this invention (see FIG. 4D) that the Dyrk1a mRNA is expressed during the differentiation into mature adipocytes. Therefore, the Dyrk1a protein might play an essential role in adipogenesis.

The expression of Dyrk1a in metabolic active tissues of wild type mice, as well as the regulation of Dyrk1a in different animal models used to study metabolic disorders, suggests that this gene plays a central role in energy homeostasis.

Taqman analysis revealed that Dyrk1b is expressed in several mammalian tissues, showing highest level of expression in testis, and higher levels in further tissues, e.g. white adipose tissue (WAT), brown adipose tissue (BAT), muscle, hypothalamus, brain, heart, lung, and kidney. Furthermore Dyrk1b is expressed on lower but still robust levels in liver, colon, small intestine, and spleen of wild type mice as depicted in FIG. 4E. We found, for example, that the expression of Dyrk1b is down regulated in the WAT and bone marrow of genetically induced obese mice (ob/ob) compared to wild type mice. Furthermore Dyrk1b is down regulated in heart and bone marrow of fasted mice compared to wild type mice (see FIG. 4F). In wild type mice fed a high fat diet, the expression of Dyrk1b is up regulated in muscle and slightly down regulated in liver and small intestine, as depicted in FIG. 4G. We show in this invention (see FIG. 4H) that the Dyrk1b mRNA is expressed and regulated during the differentiation into mature adipocytes. Therefore, the Dyrk1b protein might play an essential role in adipogenesis.

The expression of Dyrk1b in metabolic active tissues of wild type mice, as well as the regulation of Dyrk1b in different animal models used to study metabolic disorders, suggests that this gene plays a central role in energy homeostasis. This hypothesis is supported by the regulation during the differentiation from preadipocytesto mature adipocytes.

For the purpose of the present invention, it will be understood by the person having average skill in the art that any combination of any feature mentioned throughout the specification is explicitly disclosed herewith.

1. A pharmaceutical composition comprising a minibrain homologous protein or/and a functional fragment thereof, a nucleic acid molecule encoding a minibrain homologous protein or/and a functional fragment thereof or/and a modulator/effector of said nucleic acid molecule or/and said protein together with pharmaceutically acceptable carriers, diluents or/and additives.

2. The composition of claim 1, wherein the nucleic acid molecule is a vertebrate or insect minibrain nucleic acid, particulary encoding a human protein as described in Table 1, or/and a nucleic molecule which is complementary thereto or a functional fragment thereof or a variant thereof.

3. The composition of claim 1 or 2, wherein said nucleic acid molecule is selected from the group consisting of
(a) a nucleic acid molecule encoding a polypeptide as shown in Table 1, or an isoform, fragment or variant of said polypeptide;
(b) a nucleic acid molecule which comprises or is the nucleic acid molecule as shown in Table 1;
(c) a nucleic acid molecule being degenerated as a result of the genetic code to the nucleic acid sequences as defined in (a) or (b);
(d) a nucleic acid molecule that hybridizes at 50° C. in a solution containing 1×SSC and 0.1% SDS to a nucleic acid molecule as defined in claim 2 or as defined in (a) to (c) or/and a nucleic acid molecule which is complementary thereto;
(e) a nucleic acid molecule that encodes a polypeptide which is at least 85%, preferably at least 90%, more preferably at least 95%, more preferably at least 98% and up to 99.6% identical to the human protein as described in Table 1 or as defined in claim 2 or to a polypeptide as defined in (a);
(f) a nucleic acid molecule that differs from the nucleic acid molecule of (a) to (e) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis or/and the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting or/and verifying, for the treatment, alleviation or/and prevention of metabolic diseases or dysfunctions, including metabolic syndrome, obesity, or/and diabetes, as well as related disorders such as eating disorder, cachexia, hypertension, coronary heart disease, hypercholesterolemia, dyslipidemia, osteoarthritis, gallstones, or liver fibrosis, in cells, cell masses, organs or/and subjects.

14. The composition of claim 1 for application in vivo.

15. The composition of claim 1 for application in vitro.

16. Use of a nucleic acid molecule encoding a minibrain homologous protein or an isoform, a functional fragment or variant thereof, in particular a nucleic acid molecule as described in Table 1, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or polypeptide for the manufacture of a medicament for the treatment of obesity, diabetes, or/and metabolic syndrome for controlling the function of a gene or/and a gene product which is influenced or/and modified by a minibrain homologous polypeptide, particularly by a polypeptide according to claim 3.

17. Use of a nucleic acid molecule encoding a minibrain homologous protein or an isoform, a functional fragment or variant thereof, in particular a nucleic acid molecule as described in Table 1, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or said polypeptide for identifying substances capable of interacting with a minibrain homologous polypeptide, particularly with a polypeptide according to claim 3.

18. A non-human transgenic animal exhibiting a modified expression of a minibrain homologous polypeptide, particularly of a polypeptide according to claim 3.

19. The animal of claim 18, wherein the expression of the minibrain homologous polypeptide, is increased or/and reduced.

20. A recombinant host cell exhibiting a modified expression of a minibrain homologous polypeptide, particularly of a polypeptide according to claim 3.

21. The cell of claim 20 which is a human cell.

22. A method of identifying a (poly)peptide involved in the regulation of energy homeostasis or/and metabolism of triglycerides in a mammal comprising the steps of
(a) contacting a collection of (poly)peptides with a minibrain homologous polypeptide, particularly a polypeptide according to claim 3, or a functional fragment thereof under conditions that allow binding of said (poly)peptides;
(b) removing (poly)peptides which do not bind and
(b) identifying (poly)peptides that bind to said minibrain homologous polypeptide.

23. A method of screening for an agent which modulates/effects the interaction of a minibrain homologous polypeptide, particularly of a polypeptide according to claim 3, with a binding target, comprising the steps of
(a) incubating a mixture comprising
(aa) a minibrain homologous polypeptide, particularly a polypeptide according to claim 3, or a functional fragment thereof;
(ab) a binding target/agent of said polypeptide or functional fragment thereof; and
(ac) a candidate agent
under conditions whereby said polypeptide or functional fragment thereof specifically binds to said binding target/agent at a reference affinity;
(b) detecting the binding affinity of said polypeptide or functional fragment thereof to said binding target to determine an affinity for the agent; and
(c) determining a difference between affinity for the agent and the reference affinity.

24. A method for screening for an agent, which modulates/effects the activity of a minibrain homologous polypeptide, particularly of a polypeptide according to claim 3, comprising the steps of
(a) incubating a mixture comprising
(aa) said polypeptide or a functional fragment thereof;
(ab) a candidate agent
under conditions whereby said polypeptide or functional fragment thereof has a reference activity;
(b) detecting the activity of said polypeptide or functional fragment thereof to determine an activity in presence of the agent; and
(c) determining a difference between the activity in the presence of the agent and the reference activity.

25. A method of producing a composition comprising mixing a (poly)peptide identified by the method of claim 22 with a pharmaceutically acceptable carrier, diluent or/and additive.

26. The method of claim 25 wherein said composition is a pharmaceutical composition for preventing, alleviating or treating of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

27. Use of a (poly)peptide as identified by the method of claim 22 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

28. Use of a nucleic acid molecule as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

29. Use of a polypeptide as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

30. Use of a vector as defined in claim 7 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

31. Use of a host cell as defined in claim 20 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

32. Use of a minibrain homologous nucleic acid molecule or of a functional fragment thereof for the production of a non-human transgenic animal which over- or under-expresses the minibrain homologous gene product.

33. Kit comprising at least one of
(a) a minibrain homologous nucleic acid molecule or a functional fragment thereof;
(b) a minibrain homologous amino acid molecule or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or/and another modulator/effector of the nucleic acid of (a) or/and the polypeptide of (b), (e), or/and (f) and
(g) an anti-sense oligonucleotide of the nucleic acid of (a).

34. A method of producing a composition comprising mixing an agent identified by the method of claim 23 with a pharmaceutically acceptable carrier, diluent or/and additive.

35. Use of an agent as identified by the method of claim 23 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.
